(19) United States
(12) Patent Application Publication
(10) Pub. No.: US 2018/0140214 A1
(43) Pub. Date: May 24, 2018

(54) SYSTEM FOR MEASURING ELECTROMYOGRAPHIC SIGNALS OF DEEP MUSCLES BASED ON SURFACE ELECTROMYOGRAPHY

(71) Applicant: The First Affiliated Hospital, Sun Yat-sen University, Guangzhou (CN)

(72) Inventors: Shanshan Zhang, Guangzhou (CN); Xiang Huang, Guangzhou (CN); Chuhuai Wang, Guangzhou (CN); Wen Wu, Guangzhou (CN)

(21) Appl. No.: 15/782,874

(22) Filed: Oct. 13, 2017

Related U.S. Application Data
(63) Continuation of application No. PCT/CN2017/073218, filed on Feb. 10, 2017.

(30) Foreign Application Priority Data
Nov. 24, 2016 (CN) 201611056085.0

(51) Int. Cl. A61B 5/0492; A61B 5/04; A61N 1/05

(52) U.S. Cl. CPC A61B 5/0492; A61B 5/224; A61N 1/0504; A61B 5/04004

(57) ABSTRACT

The invention discloses a system for measuring electromyographic signals of deep muscles based on sEMG, comprising: a measuring electrode, an EMG signal collector and a data processing device; wherein the measuring electrode is a monopolar needle electrode or a fine-wire electrode, the EMG signal collector is connected to the measuring electrode to collect the measured EMG signals therefrom; and the data processing device is connected to the EMG signal collector for acquiring parameters corresponding to sEMG on the basis of the collected EMG signals. According to embodiments of the present invention, the electromyographic activity of a single targeted muscle and a deep muscle can be detected by replacing existing surface electrodes with monopolar needle electrodes or fine-wire electrodes, and thereby limitations that surface electrode recordings are restricted to superficial muscle groups underneath the electrodes can be eliminated.

### CROSS-REFERENCE TO RELATED APPLICATIONS

The present application is a Continuation Application of PCT application No. PCT/CN2017/073218 filed on Feb. 10, 2017 which claims the benefit of Chinese Patent Application No. 201611056085.0 filed on Nov. 24, 2016. The contents of the above are hereby incorporated by reference.

### FIELD OF THE INVENTION

The invention relates to the technical field of measuring instruments, in particular, to a system for measuring electromyographic signals of deep muscles based on sEMG.

### BACKGROUND OF THE INVENTION

Nowadays, clinical measurement of EMG signals mainly includes surface electromyography (sEMG) and concentric needle electrode electromyography (needle EMG), in both of which tiny potential difference generated by contraction of muscle fibers is amplified, recorded and transformed to digital signals, and then neuromuscular function status can be monitored by analyzing these digital signals. The potential difference varies with the neuromuscular structure and function, and electromyographic signals generated by contraction of different muscle fibers vary correspondingly, that is, the difference of constitution and property of muscles can be reflected in the electromyogram, which serves as a basis of surface electromyography diagnosis and neuromuscular function evaluation.

SEMG records muscle electrical activity to provide dynamic real-time measurement of neuromuscular function using surface electrodes. This technique has been extensively studied in sports medicine and rehabilitation, and in particular represents a unique technique for investigating the relationships between maximal isometric contraction and neuromuscular activation of selected muscles that is a low-cost, noninvasive and easy-to-use manner. sEMG involves a pair of circular Ag—AgCl electrodes with 1-cm diameter and an inter-electrode distance of 2 cm attached to the skin surface for detecting the EMG electromyographic signals underneath them. Surface electrodes were connected to an amplifier and signals were sampled and stored in random-access memory (RAM). After being collected by the EMG signal collector, the data stored in the RAM are transferred to a corresponding signal processing device (such as a computer or a sEMG system with data processing software) where they are processed for further analysis, consequently corresponding sEMG is produced. However, surface electrodes can only measure the electromyographic activity of a selected muscle under or surrounding the electrode which is easily affected by other muscles, and it is difficult to detect EMG signals of certain muscles, i.e., the multifidus, vastus intermedius muscle, and gastrocnemius, because they are located in deeper regions of the body. Taking the lumbar multifidus muscle as an example, it plays an important role in stabilization during functional movements and accounts for two-thirds of lower lumbar segmental stability. The deep fibers (DM) of lumbar multifidus act as a vital segmental stabilizer that allows for lateral flexion, rotation and extension, while the superficial fibers are thought to contribute to spinal compression and extension. Considering that the DM form the main component of the lumbar multifidus, altered activity in the superficial fibers offers limited prognostic value. However, it is difficult to record EMG signals in the deep muscles using sEMG to determine the neuromuscular activation patterns.

The primary electrodiagnostic test for the evaluation of radiculopathy is needle-electrode EMG because it has the highest sensitivity for detecting the electromyographic activity of a signal muscle or that of deep muscles. However, needle electrodes can be used to record low levels of electromyographic activity (the length of the conductive part is 0.1-0.5 mm), even activity of only several action potentials. As a result, in clinical practice, the needle EMG is generally used for measuring nerve conduction velocity and amplitude of electromyographic signals to evaluate the pathological status of neuromuscular system rather than performing real-time measurements of neuromuscular function. The signals (i.e., the action potential of muscle fibers) of sEMG and needle EMG are generated by the same physiological mechanisms underlying the electromyographic activity. Previous studies have shown that needle electrodes for recording EMG can be used to assess the validity of sEMG.

Therefore, a method is needed to detect EMG signals in the targeted deep muscles to define their function. Even though the detection techniques differ from surface and needle electrodes, it is feasible to use the sEMG system combined with needle electrodes or fine-wire electrodes for evaluating the neuromuscular activation of deep muscles.

### SUMMARY OF THE INVENTION

Embodiments of the invention disclose methods for measuring electromyographic signals of deep muscles based on sEMG system, through which the electromyographic activity of a single targeted muscle and deep muscles can be detected, and limitations that surface electrodes recording are restricted to superficial muscle groups underneath the electrodes can be eliminated, so it shows great value in clinical application.

Embodiments of the invention disclose methods for measuring electromyographic signals of deep muscles based on sEMG system, comprising: a measuring electrode, an EMG signal collector and a data processing device; wherein the measuring electrode is a monopolar needle electrode or a fine-wire electrode; the EMG signal collector is connected to the measuring electrode to collect the measured EMG signals therefrom; and the data processing device is connected to the EMG signal collector for acquiring parameters corresponding to sEMG on the basis of the collected EMG signals.

Furthermore, the monopolar needle electrode comprises a head part, a body part and a shank part; the head part and the shank part are conductive while the body part is nonconductive.

Further, the diameter of the monopolar needle electrode ranges from 0.25 mm to 0.65 mm and the length thereof ranges from 20 mm to 75 mm, wherein the length of the head part of the monopolar needle electrode ranges from 1 mm to 20 mm.

Particularly, the connection between the measuring electrode and the EMG signal collector is accomplished by connecting the shank part of the monopolar needle electrode to the EMG signal collector via an alligator clip cable or to a cable of the EMG signal collector via a connector.

Further, the fine-wire electrode is any of a copper fine-wire electrode, a platinum fine-wire electrode and a steel fine-wire electrode.

Further, the diameter of the fine-wire electrode ranges from 0.08 mm to 0.50 mm.

Particularly, the connection between the measuring electrode and the EMG signal collector is accomplished by connecting fine-wire of the fine-wire electrode to the EMG signal collector via an alligator clip cable or to a cable of the EMG signal collector via a connector.

Advantages can be achieved by implementing embodiments of the invention.

According to the present invention, the method for measuring electromyographic signals of deep muscles based on sEMG system comprises a measuring electrode, an EMG signal collector and a data processing device, wherein the measuring electrode is a monopolar needle electrode or a fine-wire electrode. Compared to the technique in prior art where sEMG is obtained by measuring electromyographic signals through surface electrodes, the technical solution of the present invention improves the electrodes in such a way that the combination of the improved needle electrode or fine-wire electrode and sEMG is able to detect the electromyographic activity of a single targeted muscle and deep muscles, and provides an effective method for selective evaluation of the activity and function status of a single targeted deep muscle. Further, the present invention uses needle electrodes and fine-wire electrodes for the measurement of EMG signals, and as such, the present invention overcomes the defect that the needle electrode can only capture a few action potentials and cannot be used to fully analyze the activity and the function status of muscles, as well as the technical prejudice in the art.

### BRIEF DESCRIPTION OF DRAWINGS

FIG. 1 is a schematic diagram of an embodiment of the system for measuring electromyographic signals of deep muscles based on sEMG according to the present invention.

FIG. 2 is a schematic diagram of an embodiment of the monopolar needle electrode according to the present invention.

FIG. 3 is a schematic diagram of an embodiment of the fine-wire electrode according to the present invention.

### DETAILED DESCRIPTION OF EMBODIMENTS

Technical solutions of embodiments of the present invention will be described clearly and completely hereinafter with reference to accompanying drawings. Obviously, the embodiments described are only part of, rather than all of the embodiments of the present invention. Any other embodiments acquired by a skilled person in this field without creative work over embodiments of the present invention shall be deemed as within the scope of the present invention.

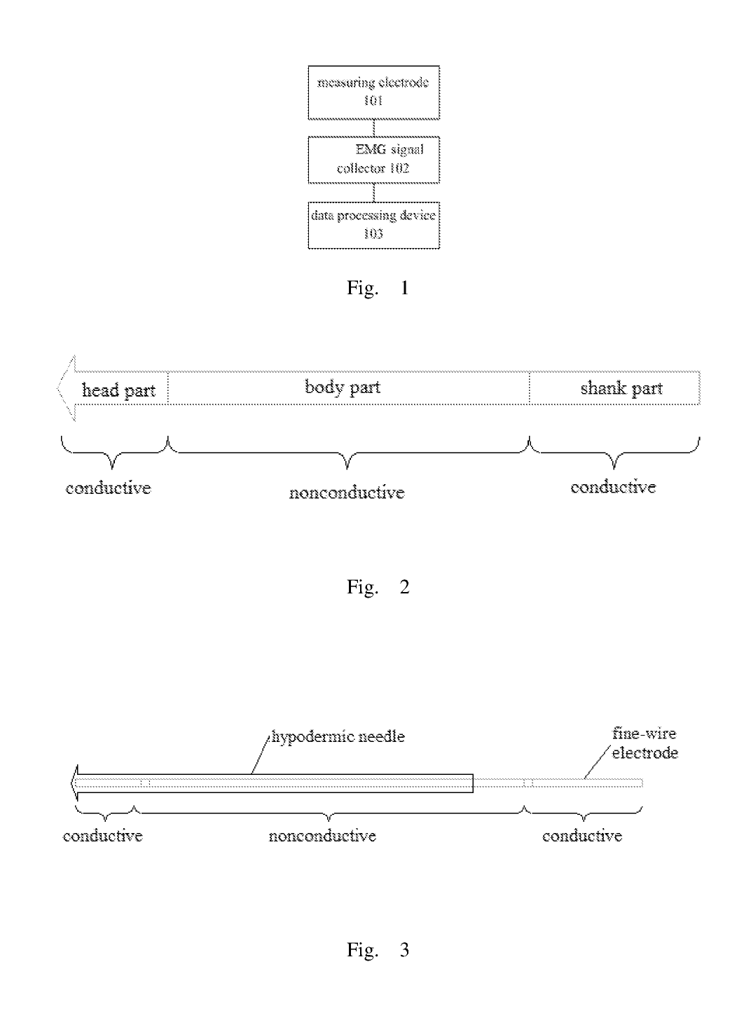

FIG. 1 illustrates a schematic diagram of an embodiment of the system for measuring electromyographic signals of deep muscles based on sEMG according to the present invention, comprising: a measuring electrode 101, an EMG signal collector 102 and a data processing device 103; wherein the measuring electrode 101 is a monopolar needle electrode or a fine-wire electrode; the EMG signal collector 102 is connected to the measuring electrode 101 to collect the measured EMG signals; the data processing device 103 is connected to the EMG signal collector 102 for acquiring parameters corresponding to sEMG on the basis of the collected signals.

In the embodiment, the EMG signal collector 102 comprises several collecting cables, each cable acts as a channel and each channel has three electrodes: two recording electrodes and one reference electrode/ground electrode. The same part of a single muscle uses one channel, and bilateral muscles use two channels. Nowadays, sEMG collectors with 4 channels or 8 channels are widely used in clinical practice. However, sEMG collectors may have 16 or 20 channels, and the number of channels can be varied as required in the clinical application and research.

In the present embodiment, the data processing device 103 may be a computer or any other data processor, which is configured for processing the acquired electromyographic signals with Fast Fourier Transform (FFT) spectral analysis, extracting the parameters such as AEMG, RMS, MF and LZ complexity of the electromyographic signals when the muscle contracts, and obtaining the parameters such as median frequency slope (MFs), and mean power frequency slope (MPFs) through analysis, and then statistic analysis is carried on the data by using statistic software like SPSS in corresponding manner, including intra-group comparison, inter-group comparison, correlation analysis and so on, in this manner, corresponding sEMG, results and conclusion are obtained. Since the data processing method is well known in prior art, detailed description thereabout is omitted.

In the present embodiment, a schematic diagram of an embodiment of the monopolar needle electrode according to the present invention is illustrated in FIG. 2. As shown in FIG. 2, the monopolar needle electrode comprises a head part, a body part and a shank part; wherein the head part and the shank part are conductive while the body part is coated with insulating varnish.

In the present embodiment, the head part of the monopolar needle electrode is configured for collecting electromyographic signals and the shank part is electrically connected to the EMG signal collector 102. The body part is coated with insulating varnish so that the interference from the electromyographic signals of superficial muscles can be avoided, in this manner, the function of the muscle being measured is specially analyzed according to the variation of the electromyographic signals of deep muscles, and the neuromuscular impairment is determined. Besides, the function regression status can be observed via the comparison between before and after treatment and thereby the curative effect can be assessed.

In the present embodiment, the diameter of the monopolar needle electrodes ranges from 0.25 mm to 0.65 mm, and it is preferable to select the electrode with a small diameter as far as possible in order to reduce the insertion pain when the head part of the monopolar needle electrode punctures skin (local anesthesia can be realized by applying a thick layer of compound lidocaine ointment on the skin surface of the muscle to be measured before the experiment). The length of the monopolar needle electrode ranges from 20 mm to 75 mm, which can be determined on the basis of the anatomical position, pattern and structure of the muscle. The length of the exposed head part of the monopolar needle electrode ranges from 1 mm to 20 mm, which can be determined by the pattern and thickness of the muscle to be measured. The length of the shank part is adaptive for connection to the alligator clip cable.

In the present embodiment, the connection between the measuring electrode 101 and the EMG signal collector 102 is accomplished by connecting the shank part of the monopolar needle electrode to the EMG signal collector 102 via an alligator clip cable or to a cable of the EMG signal collector 102 via a connector.

In the embodiment, using the improved monopolar needle electrode as a measuring electrode has the following advantages: 1. the conductive head part of the monopolar needle electrode for collecting electromyographic signals is longer, so that it can collect more action potentials for the analysis on the function status of the muscle; 2. the body part is insulated so that the interference from the electromyographic signals of superficial muscles can be avoided, in this manner, the function of the muscle being measured is specially analyzed according to the variation of the electromyographic signals of deep muscles, and the neuromuscular impairment is determined, besides, the function regression status can be observed via the comparison between before and after treatment and thereby the curative effect can be assessed; 3. The electrodes contact the muscle directly, so they reflect the frequency parameters of the muscle more sensitively than the surface electrodes, and the function status of muscles can be reflected more directly and effectively; 4. The combination of the sEMG system and the improved monopolar needle electrodes provides an effective tool for evaluating the electromyographic activity and neuromuscular function of a single targeted deep muscle, and thereby limitations that surface electrode recordings are restricted to superficial muscle groups underneath the electrodes can be eliminated.

Meanwhile, a monopolar needle electrode with a conductive surface on body part can be utilized to collect electromyographic signals of superficial muscles. The parameters of this kind of needle electrode in time domain are completely correlative with the surface electrode, but the parameters thereof in frequency domain can reflect the change of spectral features more accurately than the surface electrodes, and the result of the non-linear analysis is identical with that of the surface electrode. In clinical practice, disposable sterile acupuncture needles are adaptive for that purpose because they are easily available and less costly, so they are valuable in clinical practice (acupuncture needles with various diameters and lengths can be selected according to the muscle to be measured). The flow chart is the same with the improved monopolar needle electrode except that no improvement is carried on the needle electrode.

As shown in FIG. 3, which is a schematic diagram of an embodiment of the fine-wire electrode according to the present invention, the fine-wire electrode comprises: a disposable hypodermic needle and a fine-wire electrode placed inside the disposable hypodermic needle. After the location of the recording electrodes was verified, the needles were then withdrawn, leaving the fine-wire electrode in the deepest portion of the targeted muscle. Besides the advantage of improved needle electrodes, fine-wire electrodes can reduce the insertion pain caused by the needle electrodes during muscle contraction. It is valuable used in clinical practice for the subject of the fine-wire electrodes collecting the EMG signals almost experiences no pain during muscle contraction.

In the present embodiment, the fine-wire electrodes may be but not limited to any of fine-wire electrodes, platinum fine-wire electrodes or steel fine-wire electrodes. The electrodes should have low resistance and high sensitivity and have no impact on output impulse waves, medium and low frequency waves. The diameter of the electrode fine-wire ranges from 0.08 mm to 0.5 mm, and a stainless steel fine-wire with a diameter of 0.16 mm is recommended for its flexibility and suitable stiffness. In addition, the fine-wire electrode can be an enamelled fine-wire with an insulating layer, where some of the insulating layer can be removed as needed to expose the conductive part under the layer.

In the embodiment, the fine-wire of the fine-wire electrode can be improved in the following way: the leading end is configured to be conductive to collect electromyographic signals, the medium part is configured to be insulated to avoid interference from the electromyographic signals of superficial muscles, and the tail end is configured to be conductive for connection to the EMG signal collector 102.

In the present invention, the diameter of the fine-wire electrode ranges from 0.25 mm to 0.65 mm, and it is preferable to select the head part of the needle with a smaller diameter as far as possible in order to reduce the insertion pain when the head part punctures skin (local anesthesia can be realized by applying a thick layer of compound lidocaine ointment on the skin surface of the muscle to be measured before the experiment), the length of the disposable hypodermic needle ranges from 20 mm to 75 mm, which can be determined on the basis of the anatomical position, pattern and structure of the muscle. The disposable hypodermic needle is graduated in order to facilitate the determination of depth.

In the present embodiment, the fine-wire electrode is used in the following way: the disposable hypodermic needle is used to lead the electrode fine-wire placed therein into the deep muscle, so that the recording electrode advances into the deep muscle to be measured at a predetermined position (the depth is determined on the basis of the anatomical position, normally 2-5 cm), and after the position of the fine-wire electrode is determined via ultrasound the needle is withdrawn (the length of the fine-wire electrode exposed out of the skin is measured before and after the needle is withdrawn in order to ensure that no displacement occurs for the fine-wire electrode).

The ground electrode is inserted by 1.5-2.0 cm vertically and thereby the electrode fine-wire is leaded in. The distance between the two recording electrodes is 0.5-1 cm and the ground electrode is positioned outside of the recording electrodes by 3-5 cm.

In order to better illustrate the solution of the present invention, the detailed steps of the measurement combing sEMG with needle electrodes are as follows: 1. preparing improved needle electrodes; 2. posturing the subject, positioning and remarking the points for electrodes; 3. performing anesthesia on skin surface by applying compound lidocaine ointment; 4. sterilizing the skin with an alcohol pad; 5. puncturing skin with the needle electrode until it enters the deep muscle to be measured; 6. verifying the location of the needle electrode by ultrasound; 7. connecting the electrodes to the connecting lines of the electromyograph for sEMG; 8. detecting EMG signals in the resting state and during contraction (including maximum voluntary isometric contraction and different degrees of contraction), such as maximum strength, strength endurance and so on; 9. processing the EMG signals using sEMG analysis-feedback instrument system software and extracting parameters in time domain and frequency domain and nonlinear parameters; 10. analyzing these collected data with a statistical software like SPSS in a corresponding statistical approach to get an EMG result and an conclusion.

In order to better illustrate the solution of the present invention, the detailed steps of the measurement combing sEMG with fine-wire electrodes are as follows: 1. preparing improved fine-wire electrodes (placing fine-wire electrode inserted in a disposable hypodermic needle); 2. posturing the subject, positioning and remarking the points for electrodes; 3. performing anesthesia on skin surface by applying compound lidocaine ointment; 4. sterilizing the skin with an alcohol pad; 5. puncturing skin with the improved fine-wire until it enters the deep muscle to be measured; 6. verifying the location of electrode by ultrasound and measuring the length of the fine-wire electrode out of the skin; 7. ensuring the fine-wire electrode staying still, and then withdrawing the needles and leaving the fine-wire electrodes in the deepest portion of the targeted muscle; 8. measuring the length of the fine-wire electrode out of the skin again to ensure no displacement happens to the fine-wire electrode; 9. attaching external fine-wires to the skin using hypoallergenic medical tape, and connecting the bare end of the electrodes to amplifiers with alligator clip electrodes, and then to the connecting lines of the electromyograph for sEMG (i.e. the EMG signal collector and the data processor); 10. detecting EMG signals in the resting state and during contraction (including maximum voluntary isometric contraction and different degrees of contraction), such as maximum strength, strength endurance and so on; 11. processing the EMG signals using sEMG analysis-feedback instrument system software and extracting parameters in time domain and frequency domain and nonlinear parameters; 12. analyzing these collected data with a statistical software like SPSS in a corresponding statistic approach to get an EMG result and an conclusion.

In conclusion, embodiments of the invention disclose a system for measuring deep electromyographic signals based on sEMG, comprising: a measuring electrode 101, an EMG signal collector 102 and a data processing device 103; wherein the measuring electrode is a monopolar needle electrode or a fine-wire electrode. Compared to prior art in which sEMG is obtained by measuring electromyographic signals through surface electrode, the technical solution of the present invention improves the electrodes in such a way that the combination of the improved needle electrode or fine-wire electrode and sEMG is able to detect the electromyographic activity of a single targeted muscle and deep muscles, which provides an effective method for selective evaluation of the activity and function status of a single targeted deep muscle. Further, the present invention uses needle electrodes and fine-wire electrodes for the measurement of sEMG, and as such, the present invention overcomes the defect that the needle electrode can only capture a few action potentials and cannot fully analyze the activity and the function status of muscles, as well as the technical prejudice in the art.

Hereinbefore described are only preferred embodiments of the present invention, which do not mean any limit to the scope of the protection of the present invention. It should be noted that any modification or improvement carried out by one skilled in the art within the principle of the present invention should be taken as within the scope of protection of the present invention.

What is claimed is:

1. A system for measuring electromyographic signals of deep muscles based on sEMG, comprising: a measuring electrode, an EMG signal collector and a data processing device; wherein the measuring electrode is a monopolar needle electrode or a fine-wire electrode; the EMG signal collector is connected to the measuring electrode to collect the measured EMG signals therefrom; and the data processing device is connected to the EMG signal collector for acquiring parameters corresponding to sEMG on the basis of the collected EMG signals.

2. The system for measuring electromyographic signals of deep muscles based on sEMG according to claim 1, wherein the monopolar needle electrode comprises a head part, a body part and a shank part; the head part and the shank part are conductive while the body part is nonconductive.

3. The system for measuring electromyographic signals of deep muscles based on sEMG according to claim 2, wherein the diameter of the monopolar needle electrode ranges from 0.25 mm to 0.65 mm and the length thereof ranges from 20 mm to 75 mm, wherein the length of the head part of the monopolar needle electrode ranges from 1 mm to 20 mm.

4. The system for measuring electromyographic signals of deep muscles based on sEMG according to claim 2, wherein the connection between the measuring electrode and the EMG signal collector is accomplished by connecting the shank part of the monopolar needle electrode to the EMG signal collector via an alligator clip cable or by connecting the shank part to a cable of the EMG signal collector via a connector.

5. The system for measuring electromyographic signals of deep muscles based on sEMG according to claim 3, wherein the connection between the measuring electrode and the EMG signal collector is accomplished by connecting the shank part of the monopolar needle electrode to the EMG signal collector via an alligator clip cable or to a cable of the EMG signal collector via a connector.

6. The system for measuring electromyographic signals of deep muscles based on sEMG according to claim 1, wherein the fine-wire electrode is any of a copper fine-wire electrode, a platinum fine-wire electrode or a steel fine-wire electrode.

7. The system for measuring electromyographic signals of deep muscles based on sEMG according to claim 1, wherein the diameter of the fine-wire electrode ranges from 0.08 mm to 0.50 mm.

8. The system for measuring electromyographic signals of deep muscles based on sEMG according to claim 6, wherein the connection between the measuring electrode and the EMG signal collector is accomplished by connecting fine-wire of the fine-wire electrode to the EMG signal collector via an alligator clip cable or to a cable of the EMG signal collector via a connector.

9. The system for measuring electromyographic signals of deep muscles based on sEMG according to claim 7, wherein the connection between the measuring electrode and the EMG signal collector is accomplished by connecting fine-wire of the fine-wire electrode to the EMG signal collector via an alligator clip cable or to a cable of the EMG signal collector via a connector.
